(19) United States
(12) Patent Application Publication
(10) Pub. No.: US 2010/0282983 A1
(43) Pub. Date: Nov. 11, 2010

(54) SYSTEMS AND METHODS FOR POSITIONING PATIENTS DURING TRACKING OF TARGETS IN RADIATION THERAPY AND OTHER MEDICAL APPLICATIONS

(76) Inventors: J. Nelson Wright, Mercer Island, WA (US); Edward J. Vertatschitsch, Bellevue, WA (US)

Correspondence Address: PERKINS COIE LLP;PATENT-SEA, P.O. BOX 1247, SEATTLE, WA, 98111-1247, US

(21) Appl. No.: 12/669,024

(22) PCT Filed: Jul. 14, 2008
(86) PCT No.: PCT/US2008/070015

§ 371 Date: Jul. 28, 2010

Related U.S. Application Data
(60) Provisional application No. 60/949,695, filed on Jul. 13, 2007.

(51) Int. Cl. A61N 5/00; G06F 15/00; G01N 23/00; G05D 3/12

(52) U.S. Cl. 250/491.1; 702/150; 250/492.1; 700/302

(57) ABSTRACT

One embodiment of such a system comprises a sensor assembly mounted in a fixed location relative to a machine isocenter. According to another embodiment, the sensor assembly includes a fixed mounting bracket and an articulating arm allowing movement of the sensor assembly while allowing translation of the sensor assembly location with reference to the machine isocenter According to still another embodiment, the sensor assembly is positioned in a fixed relationship relative to the patient support assembly, mounted below the patient support assembly, as an overlay on the patient support assembly or integral to and forming a portion of the patient support assembly. The sensor assembly location is referenced to the machine isocenter through the relation of the table to the machine isocenter or through an independent locating system for the sensor assembly. According to yet another embodiment, the sensor assembly is located relative to machine isocenter by conventional imaging techniques.

### CROSS-REFERENCE TO RELATED APPLICATION

This application claims benefit of U.S. Patent Application No. 60/949,695, filed Jul. 13, 2007, titled “AN ELECTROMAGNETIC PATIENT POSITIONING SYSTEM” which is hereby incorporated by reference in its entirety.

### TECHNICAL FIELD

This invention relates generally to radiation therapy systems, and more particularly to systems and methods for accurately locating and tracking a target in a body to which guided radiation therapy is delivered.

### BACKGROUND OF THE INVENTION

Radiation therapy has become a significant and highly successful process for treating prostate cancer, lung cancer, brain cancer and many other types of localized cancers. Radiation therapy procedures generally involve (a) planning processes to determine the parameters of the radiation (e.g., dose, shape, etc.), (b) patient setup processes to position the target at a desired location relative to the radiation beam, (c) radiation sessions to irradiate the cancer, and (d) verification processes to assess the efficacy of the radiation sessions. Many radiation therapy procedures require several radiation sessions (i.e., radiation fractions) over a period of approximately 5-45 days.

To improve the treatment of localized cancers with radiotherapy, it is generally desirable to increase the radiation dose because higher doses are more effective at destroying most cancers. Increasing the radiation dose, however, also increases the potential for complications to healthy tissues. The efficacy of radiation therapy accordingly depends on both the total dose of radiation delivered to the tumor and the dose of radiation delivered to normal tissue adjacent to the tumor. To protect the normal tissue adjacent to the tumor, the radiation should be prescribed to a tight treatment margin around the target such that only a small volume of healthy tissue is irradiated. For example, the treatment margin for prostate cancer should be selected to avoid irradiating rectal, bladder and bulbar urethral tissues. Similarly, the treatment margin for lung cancer should be selected to avoid irradiating healthy lung tissue or other tissue. Therefore, it is not only desirable to increase the radiation dose delivered to the tumor, but it also desirable to mitigate irradiating healthy tissue.

One difficulty of radiation therapy is that the target often moves within the patient either during or between radiation sessions. For example, the prostate gland moves within the patient during radiation treatment sessions because of respiration motion and/or organ filling/emptying (e.g., full or empty bladder). Tumors in the lungs also move during radiation sessions because of respiration motion and cardiac functions (e.g., heartbeats and vasculature constriction/expansion). To compensate for such movement, the treatment margins are generally larger than desired so that the tumor does not move out of the treatment volume. This is not a desirable solution because the larger treatment margins may irradiate a larger volume of normal tissue.

Another challenge in radiation therapy is accurately aligning the tumor with the radiation beam. Current setup procedures generally align external reference markings on the patient with visual alignment guides for the radiation delivery device. For an example, a tumor is first identified within the patient using an imaging system (e.g., X-ray, computerized tomography (CT), magnetic resonance imaging (MRI), or ultrasound system). The approximate location of the tumor relative to two or more alignment points on the exterior of the patient is then determined. During setup, the external marks are aligned with a reference frame of the radiation delivery device to position the treatment target within the patient at the beam isocenter of the radiation beam (also referenced herein as the machine isocenter). Conventional setup procedures using external marks are generally inadequate because the target may move relative to the external marks between the patient planning procedure and the treatment session and/or during the treatment session. As such, the target may be offset from the machine isocenter even when the external marks are at their predetermined locations for positioning the target at the machine isocenter. Reducing or eliminating such an offset is desirable because any initial misalignment between the target and the radiation beam will likely cause normal tissue to be irradiated. Moreover, if the target moves during treatment because of respiration, organ filling, or cardiac conditions, any initial misalignment will likely further exacerbate irradiation of normal tissue. Thus, the day-by-day and moment-by-moment changes in target motion have posed significant challenges for increasing the radiation dose applied to patients.

Conventional setup and treatment procedures using external marks also require a direct line-of-sight between the marks and a detector. This requirement renders these systems useless for implanted markers or markers that are otherwise in the patient (i.e., out of the line-of-sight of the detector and/or the light source). Thus, conventional optical tracking systems have many restrictions that limit their utility in medical applications.

Calypso Medical has developed a patient positioning system for use with radiation therapy which includes electromagnetic localization of implanted markers. Current configurations of the Calypso Medical system include an electromagnetic array that is positioned relatively freely in the treatment room, and localized by other means (typically with optical means).

### BRIEF DESCRIPTION OF THE DRAWINGS

FIG. 1 is a side elevation view of a tracking system for use in localizing and monitoring a target in accordance with an embodiment. Excitable markers are shown implanted in or adjacent to a target in the patient.

FIG. 2 is a schematic elevation view of the patient on a movable support table and of markers implanted in the patient.

FIG. 3 is a side elevation view of a tracking system including a sensor assembly fixedly mounted to the floor for use in localizing and monitoring a target in accordance with an embodiment.

FIG. 4 is a schematic elevation view of the patient on a support table and of markers implanted in the patient, the tracking system including a sensor assembly fixedly mounted to the floor in accordance with an embodiment.

FIG. 5 is a side elevation view of a tracking system including a sensor assembly fixedly mounted to a radiation delivery device for use in localizing and monitoring a target in accordance with an embodiment.

FIG. 6 is a schematic elevation view of the patient on a support table and of markers implanted in the patient, the tracking system including a sensor assembly fixedly mounted to the radiation delivery device in accordance with an embodiment.

FIG. 7 is a side elevation view of a tracking system including a sensor assembly fixedly mounted to a patient support for use in localizing and monitoring a target in accordance with an embodiment.

FIG. 8 is a schematic elevation view of the patient on a support table and of markers implanted in the patient, the tracking system including a sensor assembly integrated into a portion of the patient support in accordance with an embodiment.

FIG. 9 is a side elevation view of a tracking system including a sensor assembly fixedly mounted to a ceiling of the treatment room for use in localizing and monitoring a target in accordance with an embodiment.

FIG. 10 is a schematic elevation view of the patient on a support table and of markers implanted in the patient, the tracking system including a sensor assembly fixedly mounted to the ceiling of the treatment room in accordance with an embodiment.

FIG. 11 is a side elevation view of a tracking system including a sensor assembly fixedly mounted to a wall of the treatment room for use in localizing and monitoring a target in accordance with an embodiment.

FIG. 12 is a schematic elevation view of the patient on a support table and of markers implanted in the patient, the tracking system including a sensor assembly fixedly mounted to a floor of the treatment room in accordance with an embodiment.

### DETAILED DESCRIPTION

### A. Overview

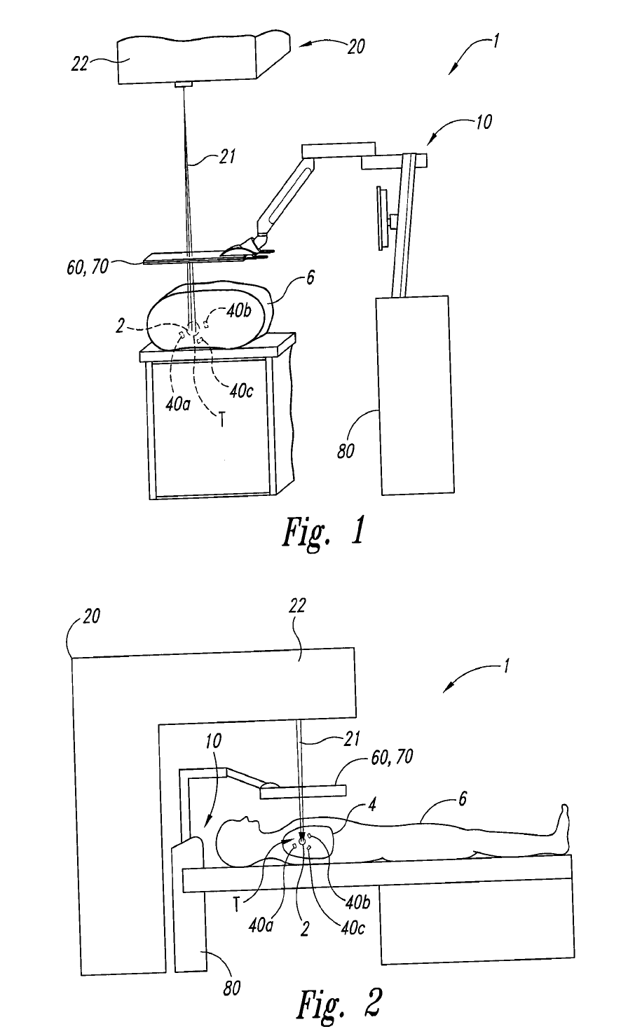

FIGS. 1-12 illustrate a system and several components for locating, tracking and monitoring a target within a patient in accordance with embodiments of the present invention. The system and components guide and control the radiation therapy to more effectively treat the target. Several embodiments of the systems described below with reference to FIGS. 1-12 can be used to treat targets in the lung, prostate, head, neck, breast and other parts of the body in accordance with aspects of the present invention. Additionally, the markers and localization systems shown in FIGS. 1-12 may also be used in surgical applications or other medical applications. Like reference numbers refer to like components and features throughout the various figures.

Under one embodiment, a system and methods are provided for accurately locating and tracking the actual position of a target within a body in preparation for and during radiation therapy. In one embodiment, the system is usable with a radiation delivery source that delivers a selected dose of radiation to the target in the body when the target is positioned at the machine isocenter of the radiation delivery source. The system includes a marker fixable in or on the body at a selected position relative to the target, such as in or near the target. The marker is excitable by an external excitation source to produce an identifiable signal while affixed in or on the body. A sensor assembly with a plurality of sensors is provided external of the body, and the sensors are spaced apart in a known geometry relative to each other. According to one embodiment, the sensor assembly is mounted in a fixed location relative to the machine isocenter. According to another embodiment, the sensor assembly includes a fixed mounting bracket and an articulating arm to allow movement of the sensor assembly while allowing translation of the location of the sensor assembly with reference to the machine isocenter. According to still another embodiment, the sensor assembly is positioned in a fixed relationship relative to the patient support assembly, for example, mounted below the patient support assembly, as an overlay on the patient support assembly, or integral to and forming a portion of the patient support assembly. According to aspects of this embodiment, the sensor assembly location is referenced to the machine isocenter through the relation of the table to the machine isocenter or through an independent locating system for the sensor assembly (i.e. an optical system). According to yet another embodiment, the sensor assembly is located relative to machine isocenter by conventional imaging techniques (e.g. x-ray).

Under yet another embodiment, an adjustable patient support assembly may further be combined with the tracking and monitoring system for use with the radiation delivery system. According to aspects of this embodiment, the support assembly includes a base, a support structure movably attached to the base, and a movement control device connected to the support structure in order to selectively move the support structure relative to the base. The sensor assembly is coupled to the base in a fixed location relative to the base. A controller is coupled to the sensor assembly to receive the signal measurement data from one or more markers in or next to the target. The controller is configured to identify the location of the target isocenter relative to the machine isocenter. The movement control device may be further coupled to the controller and adapted to position the target isocenter coincident with the machine isocenter in response to data from the controller.

Various embodiments of the invention are described to provide specific details for a thorough understanding and enabling description of these embodiments. A person skilled in the art, however, will understand that the invention may be practiced without several of these details, or that additional details can be added to the invention. Where the context permits, singular or plural terms may also include the plural or singular term, respectively. Moreover, unless the word “or” is expressly limited to mean only a single item exclusive from the other items in reference to a list of at least two items, then the use of “or” in such a list is to be interpreted as including (a) any single item in the list, (b) all of the items in the list, or (c) any combination of items in the list. Additionally, the term “comprising” is used throughout to mean including at least the recited feature(s) such that any greater number of the same feature and/or types of other features or components are not precluded.

### B. Radiation Therapy Systems with Tracking Systems

FIGS. 1 and 2 illustrate various aspects of a radiation therapy system 1 for applying guided radiation therapy to a target 2 (e.g., a tumor) within a lung 4, prostate, breast, head, neck or other part of a patient 6. The radiation therapy system 1 has a localization system 10 and a radiation delivery device 20. The localization system 10 is a tracking unit that locates and tracks the actual position of the target 2 in real time during treatment planning, patient setup, and while applying ionizing radiation to the target from the radiation delivery device. Thus, although the target 2 may move within the patient because of breathing, organ filling/emptying, cardiac functions or other internal movement as described above, the localization system 10 accurately tracks the motion of the target relative to the external reference frame of the radiation delivery device or other external reference frame outside of the patient to accurately deliver radiation within a small margin around the target. The localization system 10 can also monitor the configuration and trajectory of the marker to provide an early indicator of a change in the tumor without using ionizing radiation. Moreover, the localization system 10 continuously tracks the target and provides objective data (e.g., three-dimensional coordinates in an absolute reference frame) to a memory device, user interface, linear accelerator, and/or other device. The system 1 is described below in the context of guided radiation therapy for treating a tumor or other target in the lung of the patient, but the system can be used for tracking and monitoring the prostate gland or other targets within the patient for other therapeutic and/or diagnostic purposes.

The radiation delivery source of the illustrated embodiment is an ionizing radiation device 20 (i.e., a linear accelerator). Suitable linear accelerators are manufactured by Varian Medical Systems, Inc. of Palo Alto, Calif.; Siemens Medical Systems, Inc. of Iselin, N.J.; Elekta Instruments, Inc. of Iselin, N.J.; or Mitsubishi Denki Kabushik Kaisha of Japan. Such linear accelerators can deliver conventional single or multi-field radiation therapy, 3D conformal radiation therapy (3D CRT), inverse modulated radiation therapy (IMRT), stereotactic radiotherapy, and tomo therapy. The radiation delivery source 20 can deliver a gated, contoured or shaped beam 21 of ionizing radiation from a movable gantry 22 to an area or volume at a known location in an external, absolute reference frame relative to the radiation delivery source 20. The point or volume to which the ionizing radiation beam 21 is directed is referred to as the machine isocenter.

The tracking system includes the localization system 10 and a plurality of markers 40. The localization system 10 determines the actual location of the markers 40 in a three-dimensional reference frame, and the markers 40 are typically implanted within the patient 6. In the embodiment illustrated in FIGS. 1 and 2, more specifically, three markers identified individually as markers 40a-c are implanted in or near the lung 4 of the patient 6 at locations in or near the target 2. In other applications, a single marker, two markers, or more than three markers can be used depending upon the particular application. Two markers, for example, are desirable because the location of the target can be determined accurately and also because any relative displacement between the two markers over time can be used to monitor marker migration in the patient. The markers 40 are desirably placed relative to the target 2 such that the markers 40 are at least substantially fixed relative to the target 2 (e.g., the markers move directly with the target or at least in direct proportion to the movement of the target). The relative positions between the markers 40 and the relative positions between a target isocenter T of the target 2 and the markers 40 can be determined with respect to an external reference frame defined by a CT scanner or other type of imaging system during a treatment planning stage before the patient is placed on the table. In the particular embodiment of the system 1 illustrated in FIGS. 1 and 2, the localization system 10 tracks the three-dimensional coordinates of the markers 40 in real time relative to an absolute external reference frame during the patient setup process and while irradiating the patient to mitigate collateral effects on adjacent healthy tissue and to ensure that the desired dosage is applied to the target.

An adjustable patient support assembly may further be combined with the tracking and monitoring system for use with the radiation delivery system. According to aspects of this embodiment, the support assembly includes a base, a support structure movably attached to the base, and a movement control device connected to the support structure in order to selectively move the support structure relative to the base. The sensor assembly is coupled to the base in a fixed location relative to the base. A controller is coupled to the sensor assembly to receive the signal measurement data from one or more markers in or next to the target. The controller is configured to identify the location of the target isocenter relative to the machine isocenter. The movement control device may be further coupled to the controller and adapted to position the target isocenter coincident with the machine isocenter in response to data from the controller as further described in U.S. Publication No. 2004/0158146 A1 entitled Systems and Methods to Accurately Position Target at Beam Isocenter and/or Control Beam Depending on Target Location, herein incorporated in its entirety by reference.

### C. General Aspects of Markers and Localization Systems

As further described in US Pub. No 2006/0079764 A1 entitled Systems and Methods For Real Time Tracking of Targets in Radiation Therapy and Other Medical Applications, herein incorporated in its entirety by reference, the operation of an embodiment of the localization system 10 and markers 40a-c for treating a tumor or other target in the patient is further described. For example, the localization system 10 and the markers 40a-c are used to determine the location of the target 2 (FIGS. 1 and 2) before, during and after radiation sessions. More specifically, the localization system 10 determines the locations of the markers 40a-c and provides objective target position data to a memory, user interface, linear accelerator and/or other device in real time during setup, treatment, deployment, simulation, surgery, and/or other medical procedures. In one embodiment of the localization system, real time means that indicia of objective coordinates are provided to a user interface at (a) a sufficiently high refresh rate (i.e., frequency) such that pauses in the data are not humanly discernable and (b) a sufficiently low latency to be at least substantially contemporaneous with the measurement of the location signal. In other embodiments, real time is defined by higher frequency ranges and lower latency ranges for providing the objective data to a radiation delivery device, or in still other embodiments real time is defined as providing objective data responsive to the location of the markers (e.g., at a frequency that adequately tracks the location of the target in real time and/or a latency that is at least substantially contemporaneous with obtaining position data of the markers). Various appropriate marker configurations are further disclosed in U.S. patent application Ser. No. 10/334,700; U.S. patent application Ser. No. 09/877,498; U.S. Pat. Nos. 6,822,570, 6,812,842, and 6,835,990, herein incorporated in their entirety by reference.

1. Localization Systems

The localization system 10 includes an excitation source 60 (e.g., a pulsed magnetic field generator), a sensor assembly 70, and a controller 80 coupled to both the excitation source 60 and the sensor assembly 70. The excitation source 60 generates an excitation energy to energize at least one of the markers 40a-c in the patient 6 (FIG. 1). The excitation source 60 produces a pulsed magnetic field at different frequencies. For example, the excitation source 60 can frequency multiplex the magnetic field at a first frequency E₁ to energize the first marker 40a, a second frequency E₂ to energize the second marker 40b, and a third frequency E₃ to energize the third marker 40c. In response to the excitation energy, the markers 40a-c generate location signals L₁₋₃ at unique response frequencies. More specifically, the first marker 40a generates a first location signal L₁ at a first frequency in response to the excitation energy at the first frequency E₁, the second marker 40b generates a second location signal L₂ at a second frequency in response to the excitation energy at the second frequency E₂, and the third marker 40c generates a third location signal L₃ at a third frequency in response to the excitation energy at the third frequency E₃. In an alternative embodiment with two markers, the excitation source generates the magnetic field at frequencies E₁ and E₂, and the markets 40a-b generate location signals L₁ and L₂, respectively.

The sensor assembly 70 can include a plurality of coils to sense the location signals L₁₋₃ from the markers 40a-c. The sensor assembly 70 can be a flat panel having a plurality of coils that are at least substantially coplanar relative to each other. In other embodiments, the sensor assembly 70 may be a non-planar array of coils. According to embodiments described further herein, the sensor assembly 70 may be fixedly mounted to the floor, ceiling, wall, radiation therapy device, and/or patient support. According to alternative embodiments, the sensor assembly 70 may be mounted to an articulating arm to allow movement of the sensor assembly relative to a fixed mounting position. According to still further embodiments, the sensor assembly 70 may be fixedly or moveably integrated into the patient support. According to yet another embodiment, the sensor assembly is located relative to machine isocenter by conventional imaging techniques (e.g. x-ray).

The controller 80 includes hardware, software or other computer-operable media containing instructions that operate the excitation source 60 to multiplex the excitation energy at the different frequencies E₁₋₃. For example, the controller 80 causes the excitation source 60 to generate the excitation energy at the first frequency E₁ for a first excitation period, and then the controller 80 causes the excitation source 60 to terminate the excitation energy at the first frequency E₁ for a first sensing phase during which the sensor assembly 70 senses the first location signal L₁ from the first marker 40a without the presence of the excitation energy at the first frequency E₁. The controller 80 then causes the excitation source 60 to: (a) generate the second excitation energy at the second frequency E₂ for a second excitation period; and (b) terminate the excitation energy at the second frequency E₂ for a second sensing phase during which the sensor assembly 70 senses the second location signal L₂ from the second marker 40b without the presence of the second excitation energy at the second frequency E₂. The controller 80 then repeats this operation with the third excitation energy at the third frequency E₃ such that the third marker 40c transmits the third location signal L₃ to the sensor assembly 70 during a third sensing phase. As such, the excitation source 60 wirelessly transmits the excitation energy in the form of pulsed magnetic fields at the resonant frequencies of the markers 40a-c during excitation periods, and the markers 40a-c wirelessly transmit the location signals L₁₋₃ to the sensor assembly 70 during sensing phases.

The computer-operable media in the controller 80, or in a separate signal processor, or other computer also includes instructions to determine the absolute positions of each of the markers 40a-c in a three-dimensional reference frame. Based on signals provided by the sensor assembly 70 that correspond to the magnitude of each of the location signals L₁₋₃, the controller 80 and/or a separate signal processor calculates the absolute coordinates of each of the markers 40a-c in the three-dimensional reference frame. The absolute coordinates of the markers 40a-c are objective data that can be used to calculate the coordinates of the target in the reference frame.

2. Real Time Tracking

The localization system 10 and markers 40 enable real time tracking of the target relative to an external reference frame outside of the patient during treatment planning, set up, irradiation sessions, and at other times of the radiation therapy process. In many embodiments, real time tracking means collecting position data of the markers, determining the locations of the markers in an external reference frame (i.e., a reference frame outside the patient), and providing an objective output in the external reference frame responsive to the location of the markers. The objective output is provided at a frequency/periodicity that adequately tracks the target in real time, and/or a latency that is at least substantially contemporaneous with collecting the position data (e.g., within a generally concurrent period of time).

For example, several embodiments of real time tracking are defined as determining the locations of the markers and calculating the locations relative to an external reference frame at (a) a sufficiently high frequency/periodicity so that pauses in representations of the target location at a user interface do not interrupt the procedure or are readily discernable by a human, and (b) a sufficiently low latency to be at least substantially contemporaneous with the measurement of the location signals from the markers. Alternatively, real time means that the location system 10 calculates the absolute position of each individual marker 40 and/or the location of the target at a periodicity of approximately 1 ms to 5 seconds, or in many applications at a periodicity of approximately 10-100 ms, or in some specific applications at a periodicity of approximately 20-50 ms. In applications for user interfaces, for example, the periodicity can be 12.5 ms (i.e., a frequency of 80 Hz), 16.667 ms (60 Hz), 20 ms (50 Hz), and/or 50 ms (20 Hz). Additionally, real time tracking can further mean that the location system 10 provides the absolute locations of the markers 40 and/or the target to a memory device, user interface, linear accelerator, or other device within a latency of 10 ms to 5 seconds from the time the localization signals were transmitted from the markers 40. In more specific applications, the location system generally provides the locations of the markers 40, target, or an instrument within a latency of about 20-50 ms. The location system 10 accordingly provides real time tracking to monitor the position of the markers 40 and/or the target with respect to an external reference frame in a manner that is expected to enhance the efficacy of radiation therapy.

Alternatively, real time tracking can further mean that the location system 10 provides the absolute locations of the markers 40 and/or the target to a memory device, user interface or other device within a latency of 10 ms to 5 seconds from the time the localization signals were transmitted from the markers 40. In more specific applications, the location system generally provides the locations of the markers 40 and/or target within a latency of about 20-50 ms. The location system 10 accordingly provides real time tracking to monitor the position of the markers 40 and/or the target with respect to an external reference frame in a manner that is expected to enhance the efficacy of radiation therapy because higher radiation doses can be applied to the target and collateral effects to healthy tissue can be mitigated.

Alternatively, real-time tracking can further be defined by the tracking error. Measurements of the position of a moving target are subject to motion-induced error, generally referred to as a tracking error. According to specific embodiments, the localization system 10 and at least one marker 4 enable real time tracking of the target or other instrument relative to an external reference frame with a tracking error that is within clinically meaningful limits.

Tracking errors are due to two limitations exhibited by any practical measurement system, specifically (a) latency between the time the target position is sensed and the time the position measurement is made available, and (b) sampling delay due to the periodicity of measurements. For example, if a target is moving at 5 cm/s and a measurement system has a latency of 200 ms, then position measurements will be in error by 1 cm. The error in this example is due to latency alone independent of any other measurement errors, and is simply due to the fact that the target or instrument has moved between the time its position is sensed and the time the position measurement is made available for use. If the measurement system further has a sampling periodicity of 200 ms (i.e., a sampling frequency of 5 Hz), then the peak tracking error increases to 2 cm, with an average tracking error of 1.5 cm.

For a real time tracking system to be useful in medical applications, it is desirable to keep the tracking error within clinically meaningful limits. For example, in a system for tracking motion of a tumor or an instrument for radiation therapy, it may be desirable to keep the tracking error within 5 mm. Acceptable tracking errors may be smaller when tracking other organs for radiation therapy. In accordance with aspects of the present invention, real time tracking refers to measurement of target position and/or rotation with tracking errors that are within clinically meaningful limits.

3. Additional Embodiments of Systems for Facilitating Radiation Treatment

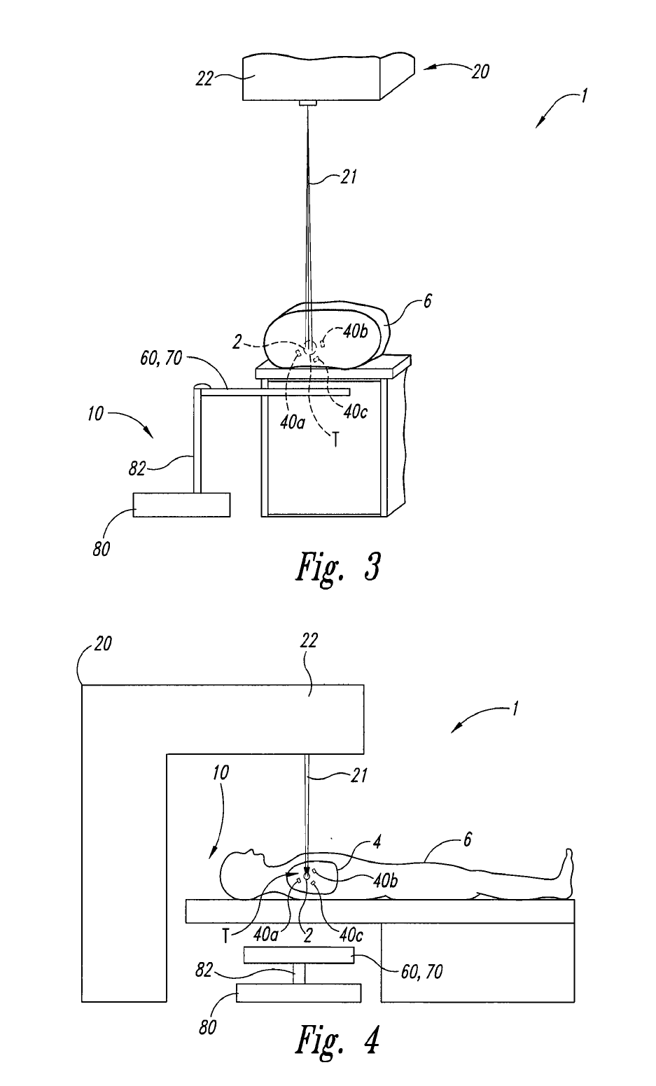

FIGS. 3-12 illustrate additional embodiments of apparatus for facilitating radiation treatment of a target in a patient. According to one embodiment, the sensor assembly is mounted in a fixed location relative to the machine isocenter. According to another embodiment, the sensor assembly includes a fixed mounting bracket and an articulating arm to allow movement of the sensor assembly while further allowing calculation of the location of the sensor assembly with reference to the machine isocenter. According to still another embodiment, the sensor assembly is positioned in a fixed relationship relative to the patient support assembly, for example, mounted below the patient support assembly, as an overlay on the patient support assembly, and/or integral to and forming a portion of the patient support assembly. According to aspects of this embodiment, the sensor assembly location is referenced to the machine isocenter through the relation of the table to the machine isocenter or through an independent locating system for the sensor assembly (i.e. an optical system). According to yet another embodiment, the sensor assembly is located relative to machine isocenter by conventional imaging techniques (e.g. x-ray).

4. Configuration of a Fixed Array Relative to the Machine Isocenter

FIGS. 3 and 4 illustrate a localization system 10 that includes an excitation source 60 (e.g. a pulsed magnetic field generator) and a sensor assembly 70 rigidly mounted to an extension 82, the extension connects the sensor assembly 70 to the controller 80. As illustrated in FIGS. 3 and 4, the controller 80 is fixedly mounted to the floor of a treatment room. According to alternative aspects of this embodiment, the extension 82 may be adjustable to pre-selected heights to accommodate adjustment of the patient support while allowing the user to maintain a known relationship between the sensor assembly 70 and the machine isocenter.

In accordance with FIGS. 3 and 4, the sensor assembly 70 is fixedly positioned in the treatment room relative to the machine isocenter. Accordingly, the sensor assembly 70 is at a fixed distance from the machine isocenter, with a known disposition. With the sensor assembly fixed in a position under the patient support as shown in FIGS. 3 and 4, the patient support can be freely adjusted laterally and longitudinally, but will have limited vertical adjustment. Alternatively, the sensor assembly 70 could be designed to be supported at multiple vertical positions, for example, by mounting the sensor assembly 70 to an adjustable extension. According to aspects of this embodiment, these positions would have to be measured or otherwise known a priori. This would enable vertical table adjustment, or enable positioning transponders closer to the sensor assembly 70 and/or excitation source 60.

The aspect of providing the sensor assembly in a fixed configuration relative to the machine isocenter, or alternatively, in a configuration with limited adjustability, is very useful because it simplifies the localization system, thus reducing overall cost and improving reliability. For example, providing the sensor assembly in a fixed configuration would allow for the elimination of a separate sensor assembly locating system (i.e. an optical system). This could allow the localization system to be used in treatment rooms that would not otherwise accommodate a localization system. Furthermore, certain treatment devices that do not require vertical positioning of the patient support may be more compatible with a simplified system. Additionally, the aspect of providing the sensor assembly in a fixed configuration relative to the machine isocenter improves the localization system accuracy by eliminating errors associated with the sensor assembly's optical localization system. Further useful aspects include simplified electromagnetic localization algorithms optimized around the known isocenter, for example, when the mounting device of the sensor assembly has a predetermined relationship to the machine isocenter.

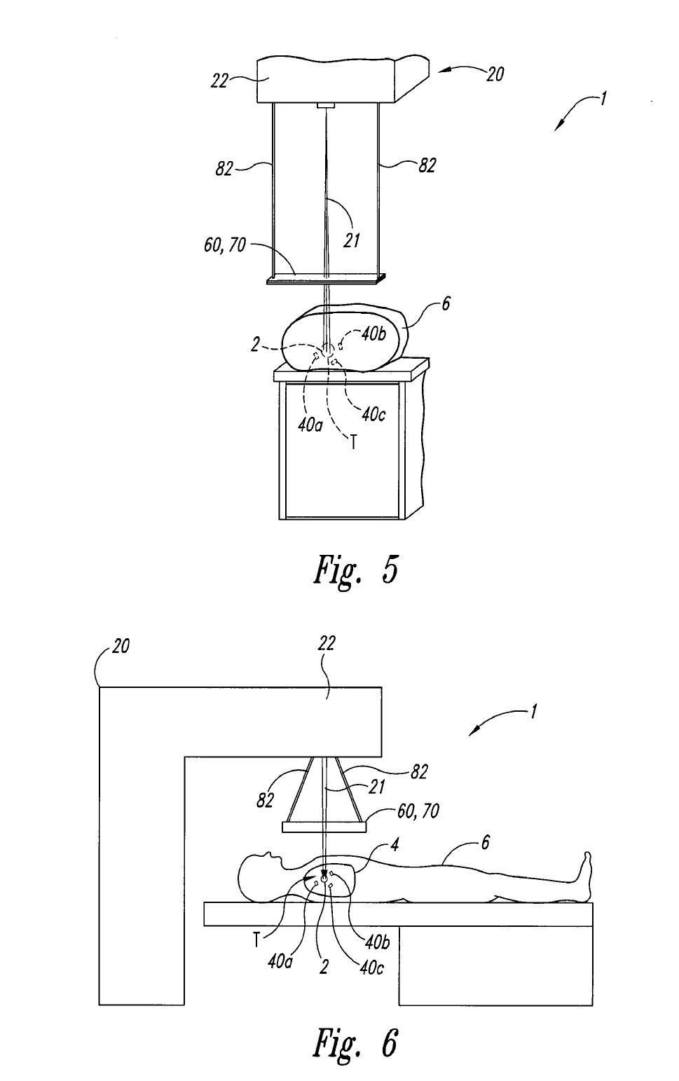

According to still further aspects of this embodiment, FIGS. 5 and 6 illustrate an excitation source 60 and sensor assembly 70 fixed to a treatment delivery device such as a linear accelerator or radiation therapy delivery device by rigid mechanical means 82. Any bracket, brace, or other mechanical means as is known in the arts may be used to rigidly affix the sensor assembly to the treatment delivery device in accordance with this disclosure. According to aspects of this embodiment, the array is fixed relative to the machine isocenter, thus an independent localization system for the sensor assembly 70 (i.e. an optical system) is not required. According to additional embodiments, the sensor assembly 70 may be moveable along the gantry 20 in a predetermined relationship relative to the machine isocenter such that the position of the array is known relative to machine isocenter.

5. Configuration of a Sensor Assembly Fixed to or in the Patient Support

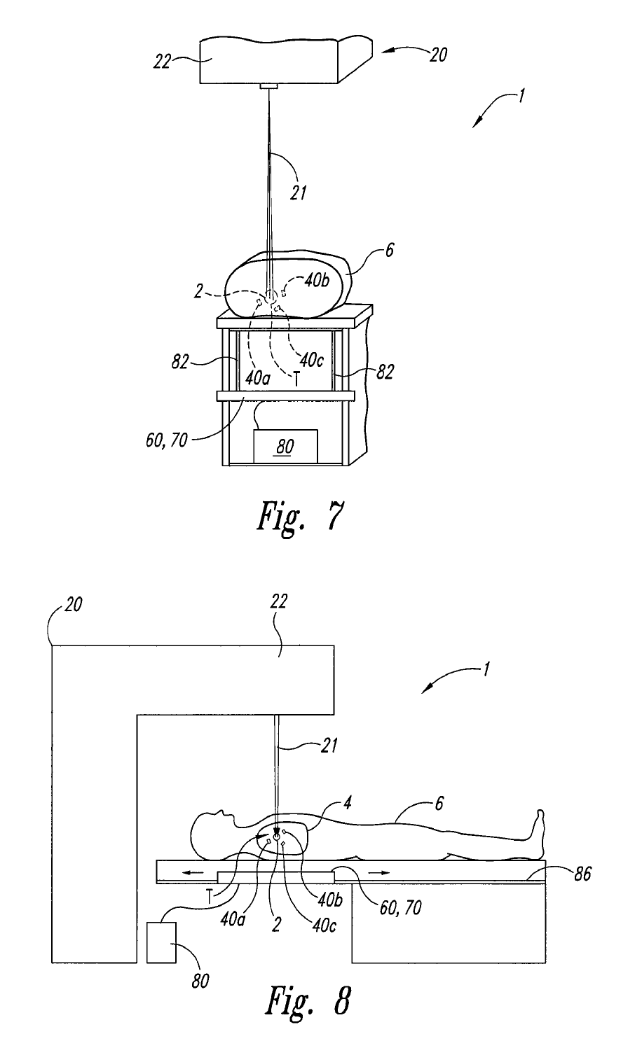

Referring to FIGS. 7 and 8, another embodiment provides a system 10 configured for use in applying guided radiation therapy to a target 12, such as a tumor, within the body 14 of a patient 16, the system including an sensor assembly 70 fixed to a patient support as shown in FIG. 7 or in a patient support as shown in FIG. 8. FIG. 7 is a side elevation view of a tracking system including a sensor assembly fixedly mounted to a patient support for use in localizing and monitoring a target in accordance with an embodiment. FIG. 8 is a schematic elevation view of the patient on a support table and of markers implanted in the patient, the tracking system including a sensor assembly integrated into a portion of the patient support in accordance with an embodiment.

In either of the embodiments shown in FIGS. 7 and 8, the sensor assembly is positioned in a fixed relationship to the patient support, but is not in a fixed relationship relative to the machine isocenter, therefore, the sensor assembly is further located relative to the machine isocenter by means of an optical system. Suitable optical systems are disclosed in U.S. Publication No. 2003/0192557, herein incorporated in its entirely. According to still a further embodiment, the sensor assembly may be located relative to isocenter by use of a magnetic locating system such as an active transponder contained on or near the sensor array and positioned in a fixed relationship to the machine isocenter. The active marker may further be mounted to the treatment device or to a fixed location in the treatment room. Alternatively, the patient support is in a fixed relationship relative to the machine isocenter and the location of the sensor assembly relative to the machine isocenter is determined from the relative position of the patient support. According to yet another alternative aspect of this embodiment, conventional imaging techniques, for example, x-ray, may be used to locate the sensor assembly relative to the machine isocenter. Once the sensor assembly is located relative to the machine isocenter, localization of the target isocenter proceeds as described above.

As shown in FIG. 8, according to one embodiment, the sensor assembly 70 may be mounted on rails 86 or other mechanical device to allow the sensor assembly 70 to move within the patient support and thus allow the user to position the sensor assembly proximate to the markers 40a, 40b, 40c.

6. Configuration of a Moveable Sensor Assembly

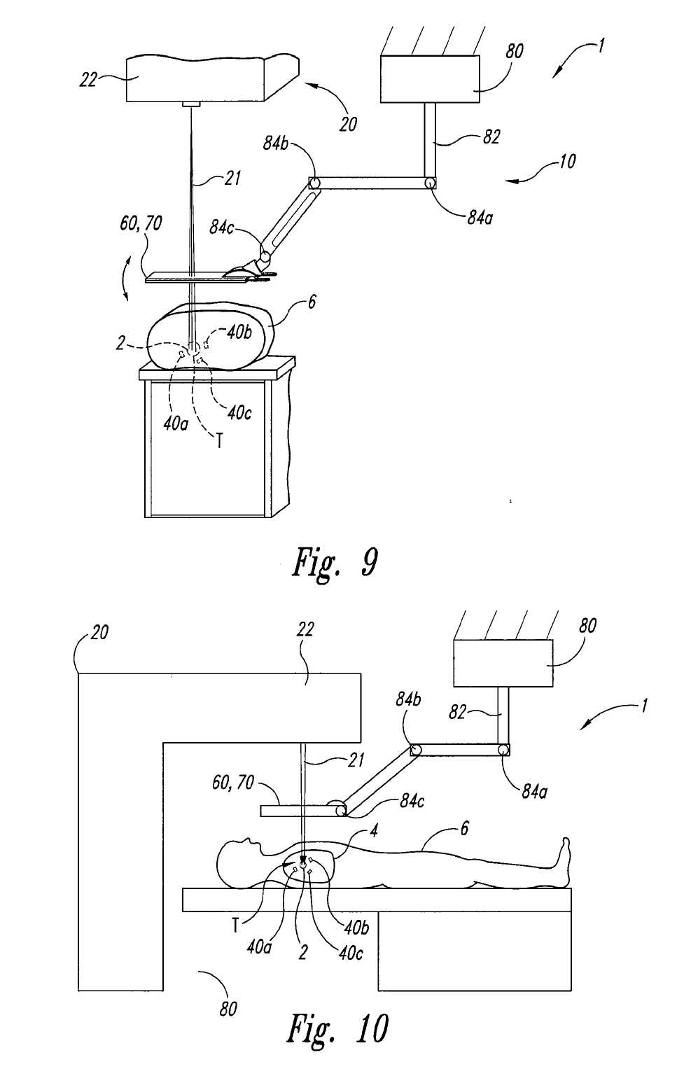

Referring to FIGS. 9-12, another embodiment provides a system 10 configured for use in applying guided radiation therapy to a target 12, such as a tumor, within the body 14 of a patient 16, the system 10 including a sensor assembly 70 configured to be moveable. According to aspects of this embodiment, the moveable sensor assembly may be fixably mounted to the ceiling, wall, floor or other fixed component of the treatment room. The fixed mounting is in a known location relative to the machine isocenter and provides a means of determining the location of the sensor assembly relative to the machine isocenter. According to one embodiment, angle encoders 84 (as are known in the art) are provided at each joint of an articulating arm 82. The angle encoders 84 allow calibration of the location of the sensor assembly 70 back to the fixed location of the controller 80 such that the location of the sensory assembly 70 can be determined relative to machine isocenter. According to another embodiment, a user interface screen may be mounted to the articulating arm, or may be mounted to a separate bracket mounted in a convenient location (not shown for purposes of clarity).

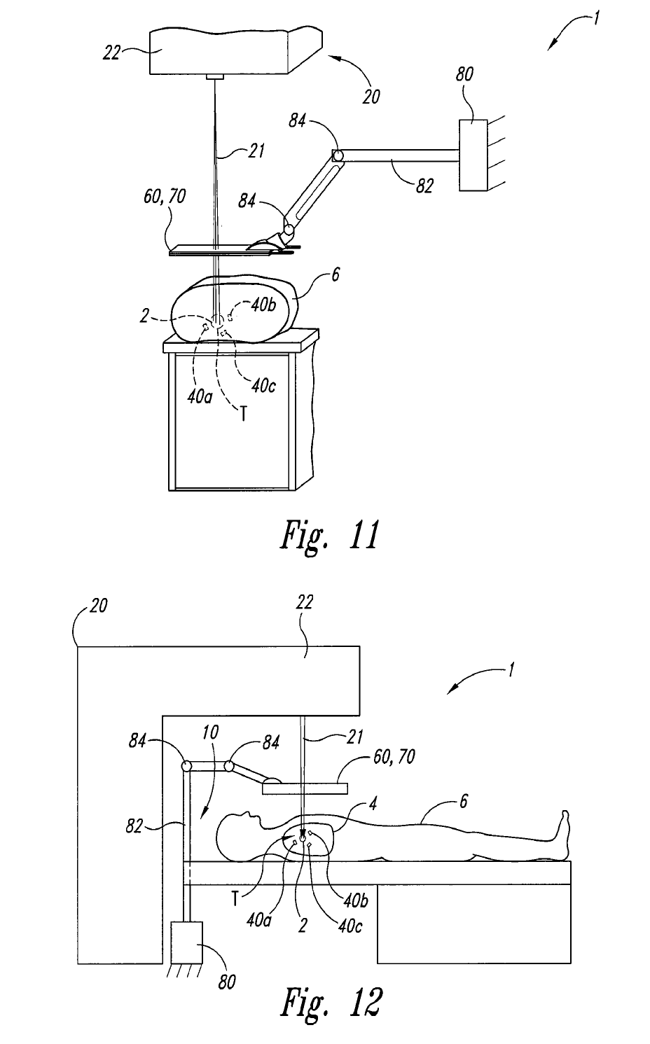

FIG. 9 illustrates a side elevation view of a tracking system including a sensor assembly fixedly mounted to a ceiling of the treatment room for use in localizing and monitoring a target. FIG. 10 illustrates a schematic elevation view of the patient on a support table and of markers implanted in the patient, the tracking system including a sensor assembly fixedly mounted to the ceiling of the treatment room. FIG. 11 illustrates a side elevation view of a tracking system including a sensor assembly fixedly mounted to a wall of the treatment room for use in localizing and monitoring a target. FIG. 12 illustrates a schematic elevation view of the patient on a support table and of markers implanted in the patient, the tracking system including a sensor assembly fixedly mounted to a floor of the treatment room.

Providing a moveable sensor assembly that includes a fixed, known mounting point and angle encoders at each joint of the articulating arm is useful to eliminate alternative means of locating the sensor assembly relative to the machine isocenter (i.e. optical systems). This embodiment thus eliminates obscuration issues relevant to optical localization systems. Additionally, this embodiment is useful in eliminating a separate console, and thus freeing up floor space. Further, if an optical system is used to locate the sensor assembly, the elimination of the console is still achieved. Providing an automated calibration of the location of the sensory assembly to the machine isocenter is useful in that it is faster, more accurate, easier to install, and/or less expensive than having a separate optical localization system for the sensor array.

According to still further embodiments, the sensor assembly may be operably connected to a movement control system, which is connected to the patient support in order to control movement of the tabletop relative to the machine isocenter. Thus, according to aspects of this embodiment, the patient support moves in response to an authorized user such as doctor, physicist or technician activating the control system, or automatically in response to instructions provided by the controller.

### D. CONCLUSION

From the foregoing, it will be appreciated that specific embodiments of the invention have been described herein for purposes of illustration, but well-known structures and functions have not been shown or described in detail to avoid unnecessarily obscuring the description of the embodiments of the invention. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of features are not precluded. It will also be appreciated that specific embodiments of the invention have been described herein for purposes of illustration, but that various modifications may be made without deviating from the inventions. For example, many of the elements of one of embodiment can be combined with other embodiments in addition to, or in lieu of, the elements of the other embodiments. Accordingly, the invention is not limited except as by the appended claims.

We claim:

1. A target locating and tracking system for use with a radiation therapy delivery machine that delivers radiation to a predetermined volume relative to a machine isocenter of the radiation therapy delivery machine, comprising:
a marker fixable at a position relative to a target in a body of a patient, the marker being excitable by an external excitation source to produce an identifiable marker signal;
a sensor assembly having a plurality of sensors arranged in a known geometry relative to each other and positioned to identify the marker signal, the sensors being configured to measure the marker signal and to provide measurement signals related to the marker signal;
a mounting device for fixedly mounting the sensor assembly at a selected position relative to the machine isocenter, the mounting device configured to be in a known position relative to the machine isocenter; and
a data processing unit coupled to the sensors to receive the measurement signals, the data processing unit being configured to use the measurement signals to determine the location of the target relative to the machine isocenter.

2. The target locating and tracking system of claim 1, further comprising a plurality of markers implantable in the body, each marker being excitable by the excitation source to produce a unique marker signal measurable by the plurality of sensors.

3. The target locating and tracking system of claim 2 wherein the plurality of markers includes at least three markers.

4. The target locating and tracking system of claim 3 wherein the markers are each axially misaligned with each other.

5. The target locating and tracking system of claim 2 wherein the marker signal from each marker has a unique frequency different from the frequency of other marker signals.

6. The target locating and tracking system of claim 1 wherein the marker is a wireless marker implantable in the body.

7. The target locating and tracking system of claim 6 wherein the data processing unit is configured to determine the position of the target relative to the sensors.

8. The target locating and tracking system of claim 1 wherein the marker is configured to be permanently implantable in the body.

9. The target locating and tracking system of claim 1 wherein the marker is a single-axis, resonating marker.

10. The target locating and tracking system of claim 1 wherein the marker is a wireless marker.

11. The target locating and tracking system of claim 1 wherein the mounting device is at a fixed location relative to the machine isocenter and further includes an articulating arm positioned between the fixed location and the sensor assembly.

12. The target locating and tracking system of claim 1 wherein the data processing unit is configured to determine (a) the location of the sensors relative to the machine isocenter and (b) the location of the markers relative to the sensors to determine the location of the target relative to the machine isocenter.

13. The target locating and tracking system of claim 1, further comprising an excitation source remote from the marker and configured to generate an excitation field that energizes the marker.

14. The target locating and tracking system of claim 1 wherein the plurality of sensors are fixed to a base in a single plane to form a sensor assembly.

15. The target locating and tracking system of claim 1, further comprising a patient support structure shaped and sized to support the body, and wherein the plurality of sensors are mounted to the patient support structure.

16. The target locating and tracking system of claim 15 wherein the table structure has a base and a tabletop, the base being in a fixed location relative to the sensors and the tabletop being movably adjustable relative to the sensors.

17. The target locating and tracking system of claim 1, further comprising a monitoring system coupled to the data processing unit, the monitoring system having a feedback portion configured to provide feedback information about the position of the target and the machine isocenter relative to each other.

18. The target locating and tracking system of claim 17 wherein the feedback portion is a visual display.

19. The target locating and tracking system of claim 17 wherein the data processing unit and monitoring system are configured to identify and display movement in real time of the target and the machine isocenter relative to each other.

20. The target locating and tracking system of claim 1 wherein the marker is one of a plurality of markers axially misaligned with each other, and the data processing unit is configured to identify a three-dimensional spatial position and orientation of the target relative to the plurality of sensors and the machine isocenter.

21. An adjustable patient support assembly for use with a radiation delivery system that delivers radiation to a machine isocenter spaced apart from the radiation delivery source, comprising:
a base;
a support structure attached to the base;
a sensor assembly having a plurality of sensors, the sensor array being carried by the support structure and/or the base, and the sensors being arranged in a known geometry relative to each other to measure a signal from an excitable marker implantable in a body at a selected position relative to a target in the body, and the sensors being configured to provide signal measurement data;
a data processing unit coupled to the sensors to receive the signal measurement data, the data processing unit being configured to use the signal measurement data to determine the location of the target relative to the machine isocenter; and
a movement control device connected to the support structure to selectively move the support structure, the movement control device being coupled to the data processing unit and movable in response to the information from the data processing unit to position the target co-incident with the machine isocenter.

22. A method of delivering radiation therapy on a selected target within a body, comprising:
translating a fixed location associated with a sensor assembly to determine the location of the sensor array relative to a machine isocenter;
implanting a leadless marker in the body at a selected position relative to the target;
exciting the implanted marker with a magnetic excitation source external of the body to produce an identifiable marker signal;
measuring the marker signal from the implanted marker with sensors positioned exterior of the body and at a known geometry relative to each other;
determining the location of the target relative to a machine isocenter of a radiation delivery assembly based on the measurements of the marker signal;
positioning the body relative to the radiation delivery device so the target is co-incident with the machine isocenter;
applying radiation from the radiation delivery device to the target and the machine isocenter; and
monitoring in real time the actual position of the target relative to the machine isocenter during application of the radiation to the target.

23. A radiation treatment planning method for establishing a therapeutic procedure for delivering ionizing radiation to a selected target, comprising:
calculating a fixed location associated with a sensor assembly relative to a machine isocenter to determine the location of the sensor array;
obtaining imaging data of a selected target within a body;
implanting an excitable marker in the body at a selected location relative to the target;
exciting the implanted marker with the external excitation source to produce the identifiable marker signal from the marker while in the body;
measuring the marker signal from the implanted marker with a plurality of sensors exterior of the body, the sensors being positioned at a known geometry relative to each other;
determining a shape and spatial orientation of the target within the body from the imaging data;
determining a target isocenter in the target within the body based upon the measurements from the sensors of the marker signal; and
developing a radiation dosage and delivery protocol for irradiating the target based upon the shape and spatial orientation of the target.

24. A method of positioning a body relative to a radiation delivery device for delivery of radiation to a target within the body, comprising:
positioning the body on a movable support assembly;
exciting an excitable marker with an excitation source exterior of the body, the marker being implanted within the body at a selected position relative to the target, the excited marker providing an identifiable marker signal;
measuring the marker signal from the implanted marker with a plurality of sensors exterior of the body, the plurality of sensors being positioned at a known geometry relative to each other and relative to the support assembly;
determining a target isocenter in the target within the body based upon the measurements from the sensors of the marker signal;
determining the location of the machine isocenter relative to a fixed location associated with a sensor assembly to determine the location of the sensor array relative to the machine isocenter;
comparing the location of the target isocenter with the location of the machine isocenter; and
moving the body relative to the machine isocenter to position the target isocenter co-incident with the machine isocenter.
